(19) United States
(12) Patent Application Publication
(10) Pub. No.: US 2021/0393579 A1
(43) Pub. Date: Dec. 23, 2021

(54) Anthocyanin and Quercetin Based Formulations for Improved Respiratory Health

(71) Applicant: Zestt Wellness Limited, Dunedin (NZ)

(72) Inventors: Darcy Marc Schack, Dunedin (NZ); Anna Wendy Campbell, Dunedin (NZ)

(73) Assignee: Zestt Wellness Limited, Dunedin (NZ)

(21) Appl. No.: 17/352,336

(22) Filed: Jun. 20, 2021

Related U.S. Application Data
(60) Provisional application No. 63/042,783, filed on Jun. 23, 2020.

(51) Int. Cl. A61K 31/353; A61K 9/00; A61K 47/46; A61K 47/44; A61K 9/107; A61K 47/26; A61K 47/36; A61K 47/12; A61K 9/06

(52) U.S. Cl. CPC A61K 31/353; A61K 9/0095; A61K 47/46; A61K 47/44; A61K 9/06; A61K 47/26; A61K 47/36; A61K 47/12; A61K 9/107

(57) ABSTRACT

A formulation includes anthocyanins and quercetin. The formulation may be implemented in a number of different ways. For example, the formulation includes a sugar phase and an anthocyanin/quercetin phase that includes the anthocyanins and the quercetin. The sugar phase and the anthocyanin/quercetin phase are based on emulsification process(es). The sugar phase is configured to facilitate combination of the anthocyanins and the quercetin including to facilitate distribution of the anthocyanins and the quercetin within the formulation. Based on the formulation being administered to a subject, the formulation is configured to perform to increase elasticity of lungs, reduce of inflammation within lungs, minimize buildup of fluid within the lungs, and facilitate removal of mucus from the lungs of the subject, reduce pneumonia thereby improving respiratory function of the lungs, and/or stop or reduce viral replication. Note that the formulation may be implementation in a gel type form or a dry type form.

### CROSS REFERENCE TO RELATED PATENTS/PATENT APPLICATIONS

The present U.S. Utility patent application claims priority pursuant to 35 U.S.C. § 119(e) to U.S. Provisional Application No. 63/042,783, entitled “Anthocyanin and Quercetin Based Formulations for Improved Respiratory Health,” (Attorney Docket No. ZST00001-01P), filed Jun. 23, 2020, which is hereby incorporated herein by reference in its entirety and made part of the present U.S. Utility patent application for all purposes.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

### Technical Field of the Invention

This invention relates generally to formulations having properties for improving health of a subject including respiratory health and more particularly to formulations that include anthocyanins and quercetin.

### Description of Related Art

People can be adversely be affected by a number of conditions with respect to their health. For example, considering respiratory related elements, people can adversely be affected by any number of conditions such as asthma, pneumonia, interstitial lung disease, acute respiratory distress syndrome (ARDS), chronic obstructive pulmonary disorder (COPD), bronchitis, etc.

Moreover, epidemics and/or pandemics that are virus based may also adversely affect the respiratory conditions of people. Nearly every year, various outbreaks of the common cold and influenza adversely affect the health of people including the respiratory health of people throughout the world. Although vaccines have been used for years to try to help stem the number of those people affected by influenza, such vaccines that may be effective one year may not be as effective in a subsequent year, given that the influenza virus changes very rapidly. Some estimates are that approximately ⅓ of the people who are infected with influenza are asymptomatic. Unfortunately, those who are infected with influenza yet asymptomatic may infect others.

Also, starting in late 2019, the COVID-19 pandemic, sometimes referred to as the coronavirus pandemic, caused by the severe acute respiratory syndrome coronavirus 2 (SARS-CoV-2) that was first identified in Wuhan, China, has produced more than 7 million cases of COVID-19 within more than 180 countries and territories. Some estimates identified more than 400,000 deaths worldwide due to the COVID-19 pandemic. Certain vaccines have been developed to reduce the likelihood of infection of a subject by the SARS-CoV-2. Estimates are that approximately 35% of the people who are infected with the SARS-CoV-2 are asymptomatic. Again, as with influenza, unfortunately, those who are infected with the SARS-CoV-2 even if asymptomatic may infect others. Oftentimes, people who are infected by a virus develop other conditions such as pneumonia, acute respiratory distress syndrome (ARDS), etc. For example, many people infected by the SARS-CoV-2 subsequently developed pneumonia and/or ARDS.

In addition, in certain parts of the world suffering from very poor air quality, pollution, etc. even those people not directly suffering from any particular respiratory ailment may experience reduced respiratory function simply due to very poor air quality, pollution, etc. in the environment in which they live. Inflammation within the body may also adversely affect the health of people. For example, inflammation within the lungs, such as constriction of the bronchial tubes within the lungs of a person may be caused for a variety of reasons. For example, certain respiratory ailments such as asthma may result in the constriction of the bronchial tubes within the lungs of a person and result in very difficult or labored breathing for that person. For example, acute or chronic inflammation of the bronchial tubes within the lungs of a person restricts the air pathways through which the air flows from the mouth and nose of the person to the alveoli (alternatively referred to as the pulmonary alveoli) within the lungs thereby resulting in difficult or labored breathing for that person. Also, certain people may be adversely affected by very poor air quality, pollution, etc. such that their immune system responds with an inflammatory response to one or more bodily portions.

Generally speaking, there are a number of variables that may adversely affect the overall health of people including their respiratory health. There continues to be an ongoing need in the art for means of treating such conditions within people to improve the health of people including their respiratory health.

### BRIEF DESCRIPTION OF THE SEVERAL VIEWS OF THE DRAWING(S)

FIG. 1A is a schematic block diagram of an embodiment of a general molecular structure of quercetin in accordance with the present invention;

FIG. 1B is a schematic block diagram of an embodiment of a general molecular structure of anthocyanin in accordance with the present invention;

FIG. 2 is a schematic block diagram of an embodiment of an emulsifier and vessel for use to generate formulations in accordance with the present invention;

FIGS. 3A and 3B are schematic block diagrams of embodiments of sugar phase preparation in accordance with the present invention;

FIGS. 4A, 4B, and 4C are schematic block diagrams of embodiments of anthocyanins/quercetin phase preparation in accordance with the present invention;

FIG. 5A is a schematic block diagram of an embodiment of a mixing vessel for use to generate formulations in a gel type form based on a sugar phase and an anthocyanin/quercetin phase in accordance with the present invention;

FIG. 5B is a schematic block diagram of an embodiment of a subject ingesting a formulation in accordance with the present invention;

FIG. 6A is a schematic block diagram of an embodiment of a system for use to generate formulations in a dry type form based on a sugar phase and the anthocyanins/quercetin phase in accordance with the present invention;

FIG. 6B is a schematic block diagram showing different examples of modes of a product within a rotating tube or cylinder for use to generate formulations in a dry type form in accordance with the present invention;

FIG. 7 is a schematic block diagram showing examples of portions of a respiratory system of a subject that may be adversely affected by one or more conditions in accordance with the present invention; and

FIG. 8 is a schematic block diagram showing an example of portions of a knee joint of a subject that may be adversely affected by one or more conditions in accordance with the present invention; and

FIGS. 9A, 9B, 10A, and 10B are schematic block diagrams of embodiments of methods for execution to by one or more devices to generate a formulation in accordance with the present invention.

### DETAILED DESCRIPTION OF THE INVENTION

This disclosure presents various novel formulations based on a combination of quercetin and one or more anthocyanins. In certain examples, as few as one type of anthocyanin is used in a formulation (e.g., including a certain amount of the one type of anthocyanin in the formulation). However, in other examples, multiple, different respective anthocyanins are used in a formulation. Note that the characteristics, aspects, properties, etc. described herein with respect to any formulation that includes a combination of quercetin and anthocyanins may be alternatively implemented based on as few as a combination of quercetin and one particular type of anthocyanin. Some formulations are based only on the combination of quercetin and anthocyanins. Also, note that other formulations include one or more additional elements in addition to the combination of quercetin and anthocyanins. In some examples, a sugar-based agent, such as honey, or some other type of sugar based agent, is used to carry the combination of quercetin and anthocyanins in a suitable form to be administered to a subject. To facilitate the quercetin being absorbed into the body of the subject, it is distributed into the sugar-based agent and specifically in very small molecules thereof. From certain perspectives, the sugar-based agent may be viewed as a means to get the quercetin into a solution to facilitate application to a subject. Generally speaking, the subject may be viewed as a person, a patient, a human, etc. to whom the formulation may be administered. Note that any such alternative and/or or equivalent nomenclature such as subject, user, person, patient, human, etc. and/or any other equivalent nomenclature may be used interchangeably.

In certain examples, the formulation is carried in a sugar-based agent as described above and is ingested orally by the subject such that the formulation enters the gastrointestinal tract of the subject and enters the subject's bloodstream via the liver. In other examples, the formulation is included in a dry type form such that it may be administered to a subject within a pill or capsule, or via a bronchial type inhaler, such that the dry type form is administered via inhalation by the subject and absorbed into the subject's bloodstream via the internal lining of the lungs of the subject.

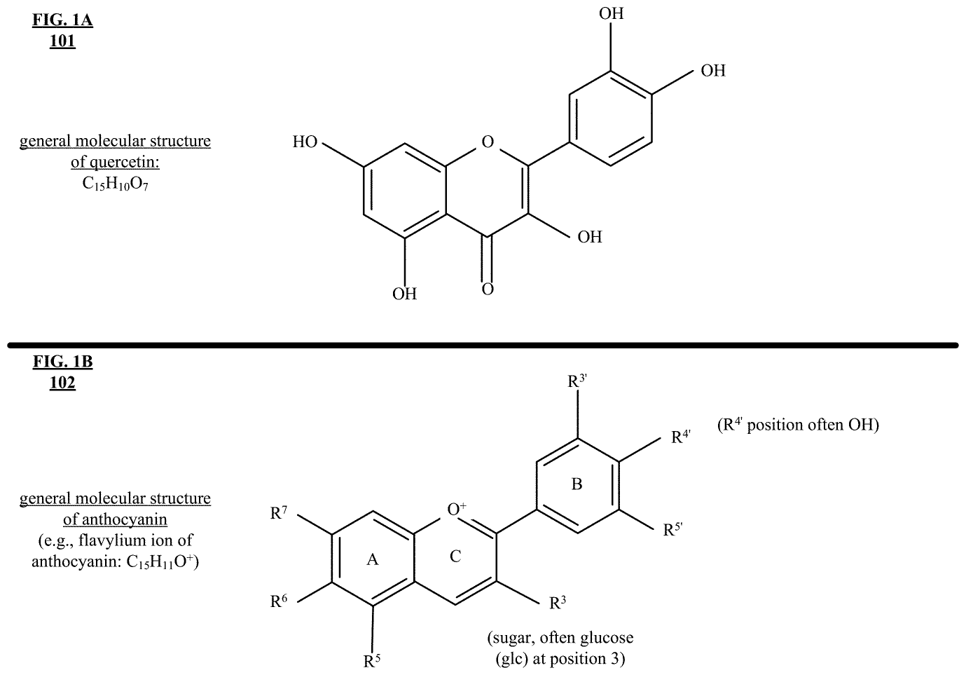

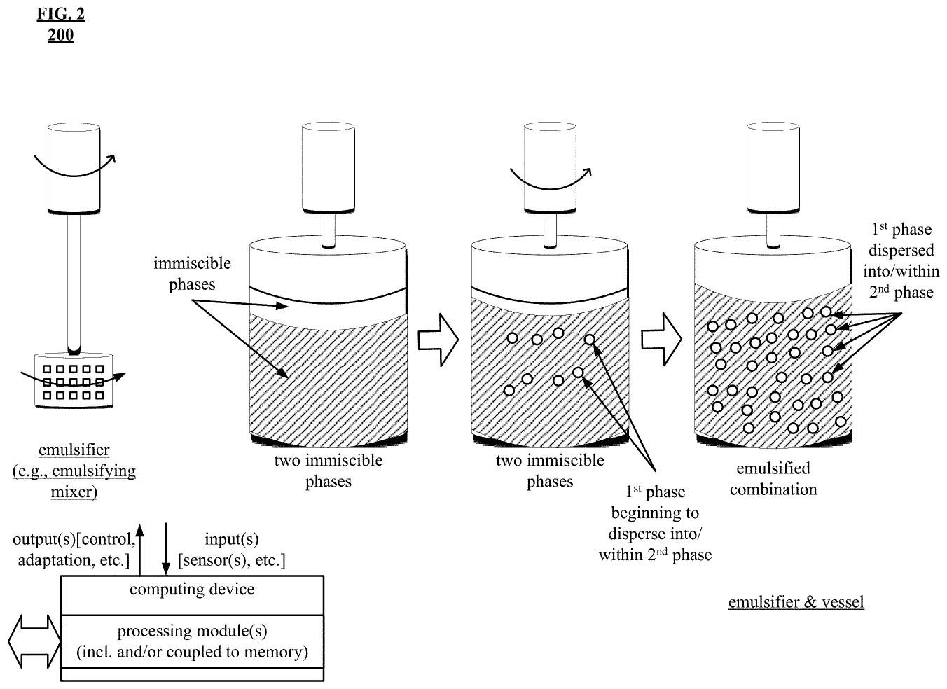

FIG. 1A is a schematic block diagram of an embodiment 101 of a general molecular structure of quercetin in accordance with the present invention. The molecular formula of quercetin is C₁₅H₁₀O₇. (i.e., a molecule composed of carbon, C, hydrogen, H, and oxygen, O). Quercetin is a plant flavonol that is included within the flavonoid group of polyphenols. Generally speaking, flavonols are a class of flavonoids that have a 3-hydroxyflavone backbone. Quercetin is found naturally within a variety of different types of foods including certain fruits, vegetables, etc. In addition, quercetin may be found within certain seeds and grains. Quercetin has properties such that, when administered to a subject, is operative to reduce inflammation within a subject and also reduce allergic reactions and effects of a subject. For example, quercetin operates to provide inhibition of the lipoxygenase and cyclooxygenase pathways. This is operative to cause a reduction or prevention of pro-inflammatory mediators within the subject thereby producing anti-inflammatory and anti-allergic effects within the subject.

FIG. 1B is a schematic block diagram of an embodiment 102 of a general molecular structure of anthocyanin in accordance with the present invention. Anthocyanins are part of a parent class of molecules called flavonoids; they are colored water-soluble pigments belonging to the phenolic group.

The word anthocyanin is derived from Greek and specifically from the two Greek words “anthos” meaning flower and “kyanous” meaning dark blue. Note that there are a variety of different types of anthocyanins. The cyanidin-3-glucoside (i.e., one of the anthocyanin pigments) is the major anthocyanin found in most of the plants. Anthocyanins appear as red but turn blue when the pH increases such as in an acidic environment. For example, in generally acidic conditions, anthocyanins appears as red pigment while blue pigment anthocyanins exists in alkaline conditions.

This diagram shows the general molecular structure of an anthocyanin. Anthocyanins belong to a parent class of molecules called flavonoids synthesizes via the phenylpropanoid pathway. They have the general molecular structure as shown in the diagram of flavylium ion that includes a lack of a ketone oxygen at the 4-position of its C-ring (carbon ring) structure. The empirical formula for flavylium ion of anthocyanins is C₁₅H₁₁O⁺. Again, this is merely possible example of anthocyanins being a flavylium ion of anthocyanin having a molecular formula of C₁₅H₁₁O⁺ (i.e., a molecule composed of carbon, C, hydrogen, H, and oxygen, O). The general molecular structure of anthocyanins are shown in the diagram with various letters R (representing various other elements) that may attach to the A, B, and C rings of the general C-ring (carbon ring) structure of the general molecular structure of an anthocyanin. These various letters R (e.g., R³, R⁴, R⁵, R⁶, and R⁷, and certain respective primes such as R^{3′}, R^{4′}, and R^{5′}) may correspond to various elements in different anthocyanins. For example, in many anthocyanins, sugar, oftentimes glucose, is included at the R³ location at the lower right portion of the general molecular structure of an anthocyanin.

Anthocyanins are one of the subclasses of phenolic phytochemicals. Anthocyanidins are often referred to in the context of the form of glycoside. When anthocyanin is in the form of glycoside, anthocyanidin is known as the aglycone. Some examples of common types of anthocyanidins include cyanidin, delphinidin, pelargonidin, peonidin, petunidin, and malvidin. For example, referring to the general molecular structure of the diagram, cyanidin includes OH (oxygen and hydrogen) at locations R³, R⁵, R⁷, R^{3′}, and R^{4′} and nothing at locations R⁶ and R^{5′}. Delphinidin includes OH (oxygen and hydrogen) at locations R³, R⁵, R⁷, and R^{4′} and nothing at locations R⁶, R^{3′}, and R^{5′}. Other variations and examples include different elements at certain locations as well.

Generally speaking, food plants that are rich in anthocyanins have relatively dark colors. Many food plants having red, blue, purple, or black color are rich in anthocyanins. Some examples of such food plants include boysenberries, blueberries, raspberries, cranberries, black raspberries, red raspberries, and blackcurrants. Even other examples include Marion blackberries, wild blueberries, cherries, redcurrant, purple corn, blue corn, red grapes, red cabbage, etc. in addition, certain red-flashed peaches and apples contain anthocyanins. Other plant foods not having such red, blue, purple, or black color may still include some amount of anthocyanins but generally much less than those that do have such red, blue, purple, or black color (e.g., bananas, potatoes, pears, etc.). When anthocyanins are ingested by the subject, the metabolites of the ingested anthocyanins are absorbed in the gastrointestinal tract of the subject and enter the subject's bloodstream via the liver of the subject.

Many of the formulations presented herein are based on a combination of quercetin and anthocyanins. The quercetin and the anthocyanins operate cooperatively with respect to improving the health of the subject. Certain health benefits that may be realized within the subject include improved respiratory function and reduced inflammation. The respiratory function of the subject is improved based on administration of such a formulation to the subject. Examples of such improvements in respiratory function of the subject may include any one or more of reduction of inflammation within the lungs, minimizing the buildup of fluid within the lungs, facilitation of the removal of mucus from the lungs, reduction of the pneumonia caused by various diseases including the severe acute respiratory syndrome coronavirus 2 (SARS-CoV-2), etc. In addition, such a formulation as presented herein exhibit properties that hinder viral replication such as may be experienced by a subject with respect to certain virus-based ailments such as the common cold, influenza, SARS-CoV-2, etc.

In addition, such a formulation is configured to increase the elasticity of the lungs of the subject. Unfortunately, as a subject ages and grows older, the elasticity of the lungs may degrade thereby causing respiratory distress for the subject. Again, there are different means by which such a formulation may be administered to a subject. When administered to a subject, the formulation is operative to improve the health of the subject regardless of the means by which the formulation is administered to enter into the subject's bloodstream (e.g., such as by being included within a product that is ingested by the subject so that the formulation enters into the subject's bloodstream via the subject's liver, such as by being included within a dry form product that is included within a pill or capsule that is swallowed by the subject and enters into the subject's bloodstream via the subject's liver, such as by being included within a dry form product that is inhaled into the lungs of the subject, etc.).

FIG. 2 is a schematic block diagram of an embodiment 200 of an emulsifier and vessel for use to generate formulations in accordance with the present invention. Certain liquids do not mix well with one another. For example, consider oil and water. Generally speaking, when the two liquids are introduced to one another, they separate from one another within a container vessel. When two liquids are generally not mixable or blendable together, they may be referred to as immiscible. However, even though certain liquids may be immiscible and not easily mixed together, certain operations may be performed on the liquids to facilitate them mixing together.

An emulsion is a mixture of such immiscible liquids that are normally unmixable or unquenchable. An emulsifier, such as an emulsifying mixer, may be used to facilitate the combination of two normally immiscible liquids. An emulsifier may be viewed as a machine that is configured to deliver a very high hydraulic shear to a liquid to facilitate it being combined with another liquid that normally doesn't mix with it. In certain examples, an emulsifier will include a motor that rotates a shaft that is connected to a mechanism including a series of teeth that are effective to shear the liquid to facilitate its combination with another liquid. For example, as this series of teeth spin within the liquid as driven by the motor, this will hydraulically force the product out with a very high force thereby breaking the particles down. This process is configured to prepare the liquid for combination with another liquid when such liquids do not generally combine with one another well (e.g., they are immiscible).

In this diagram, on the left-hand side, the motor is shown connecting to a shaft that is connected to a mechanism including a series of teeth. As the motor rotates the shaft, these teeth are configured to shear liquid in contact with those teeth. Moving to the right of the diagram, an emulsifier and vessel are shown operating in combination with one another. Considered two immiscible phases (e.g., two liquids that are generally unmixable or unblendable with one another) that are included within a vessel. As the motor begins to operate thereby rotating the shaft and the series of teeth, the two immiscible phases began to mix with one another. For example, consider the first phase beginning to disperse into the second phase such that a first phase-into-second phase emulsion is being formed. At the right hand side of the diagram, the first phase is shown as being fully dispersed into or within the second phase.

Generally speaking, emulsions are often described as including both a dispersed phase and a continuous phase. For example, consider one of the phases being dispersed into another phase. The boundary between the dispersed phase and the continuous phase is typically referred to as the interface. Note that emulsification may be performed to different degrees with respect to various liquids thereby generating different classes of emulsions. For example, the droplet size within emulsions may vary depending on the degree and intensity of emulsion that is performed. When the droplet size becomes very small, such as less than 100 nanometers (nm) in diameter, such emulsions may be referred to as micro-emulsions and/or nano-emulsions.

In an example of operation and implementation, the operation of such components in this diagram and others herein is controlled by one or more computing devices, processing modules, etc. For example, a computing device includes memory (e.g., computer readable memory) that stores operational instructions and one or more processing modules operably coupled to the memory. In certain examples, the computing device is operably coupled to an emulsifier. In certain other examples, the computing device is operably coupled to a mixing vessel. In yet other examples, the computing device is operably coupled to any one or more of an atomization spray nozzle(s), conveyor belt system(s), rotating mechanism(s), etc.

For example, the one or more processing modules is configured to execute the operational instructions to generate one or more signals (e.g., bit stream, stream, signal sequence, etc. (or their equivalents)) that are provided to the emulsifier to control the operation of the emulsifier (e.g., adjusting the speed of rotation of the emulsifier). In certain examples, the one or more processing modules is configured to execute the operational instructions to generate one or more signals that are provided to the mixing vessel to control the operation of the mixing vessel including the operational speed of the mixing vessel. In addition, note that any of a number of sensors (e.g., speed rotational sensors, pressure sensors, temperature sensors, etc.) may be implemented within the system and operably coupled and configured to provide information to the computing device for use by the computing device in governing the operation of the system (e.g., including the emulsifier and mixing vessel).

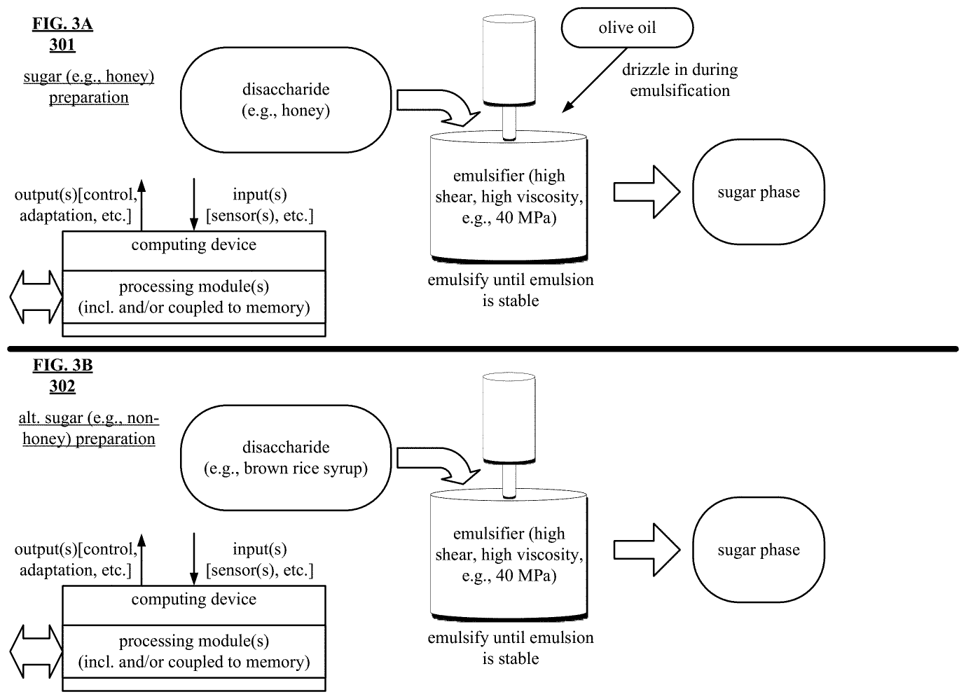

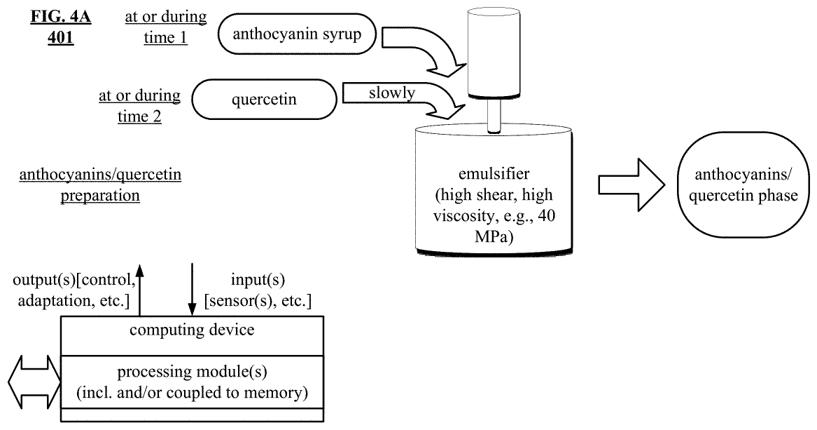

FIGS. 3A and 3B are schematic block diagrams of embodiments 301 and 201, respectively, of sugar phase preparation in accordance with the present invention.

Referring to embodiment 301 of FIG. 3A, this diagram shows an emulsifier that operates on the disaccharide to generate a sugar phase to be used to generate formulations that include anthocyanins and quercetin. In one example, the disaccharide to be honey. The honey is put into the emulsifier to undergo emulsification thereby generating the sugar phase. Generally speaking, liquid honey can have a viscosity within the 2,000 to 10,000 millipascal seconds (mPa-s) range. Note that different types of honey may have different viscosities, e.g., some higher and some lower. In one specific implementation, a disaccharide having a viscosity of approximately 8,000 mPa-s is used (e.g., an appropriately selected honey having a viscosity of approximately 8,000 mPa-s). The emulsifier operates to perform a micronization process on the honey to facilitate its combination with the anthocyanins and quercetin including to facilitate an even distribution of the anthocyanins and quercetin in the formulation. When micronization is performed on the honey, it operates to reduce the particle size of the honey (e.g., reduces the average diameter of the particles of the honey). The honey is operative to serve as a carrier for anthocyanins and quercetin for administration to a subject.

In certain examples, note that a very high shear, high viscosity emulsifier is employed. For example, an emulsifier capable of operating at a pressure of 40 megapascals (MPa) or more is suitable to perform the emulsification operation on the honey. In addition, note that most honey naturally has hydrogen peroxide in it. This hydrogen peroxide provides certain benefits for the honey including increasing its resistivity to bacteria. However, this same hydrogen peroxide may adversely affect the anthocyanins and quercetin with which the honey is combined. To address this potential adverse effect of the hydrogen peroxide on the anthocyanins and quercetin, we use a medicinal-grade Manuka Honey high which is high in methylglyoxal (MGO). Although hydrogen peroxide (H₂0₂) is one of the major factors in the majority of honey it is not present in medicinal-grade Manuka Honey and does not adversely affect the anthocyanins and quercetin, if regular honey is to be used then olive oil is drizzled into the vessel during the emulsification process being performed on the honey. For example, olive oil can have a viscosity within the range of 50 to 100 millipascals seconds (mPa-s) range. Note that different types of olive oil may have different viscosities, e.g., some higher and some lower. In one specific implementation, an olive oil having a viscosity of approximately 56 mPa-s is used. The honey is emulsified within the emulsifier until the emulsion is stable. The olive oil is added to stop the glucose oxidase in the honey from releasing hydrogen peroxide from glucose thereby eliminating or reducing any adverse effects on the anthocyanins and quercetin that may be caused by the hydrogen peroxide. In addition, the olive oil, which includes triglycerides, improves and facilitates the shearing of the honey and isolates the hydrogen peroxide therein. Then, as the quercetin is added in, the quercetin is being stuck between the particles of the hydraulically sheared honey and not damaged by the hydrogen peroxide of the honey. The olive oil operates to perform chemical or mechanical ablation of the honey that reduces or stops damage to the anthocyanins and quercetin that may be caused by the hydrogen peroxide of the honey.

As the honey is hydraulically sheared in the emulsifier, the quercetin is added (slowly) into the vessel during the emulsification process. This emulsification of the honey facilitates a substantially uniform distribution of the quercetin throughout the hydraulically sheared honey. The quercetin binds to the hydraulically sheared honey, which serves as the agent for to carry the quercetin into the body thereby facilitating improved absorption of the quercetin into the subject.

Referring to embodiment 302 of FIG. 3B, this diagram shows an alternative disaccharide, such as brown rice syrup, sugar, etc. being used as the sugar phase. Note that such alternative disaccharides made undergoing emulsification as described above with respect to the honey if desired. Note that an alternative disaccharide, such as brown rice syrup, sugar, etc. that does not naturally include hydrogen peroxide may be used. In such an instance, use of such an alternative disaccharide does not necessarily require the operation of adding/drizzling of olive oil into the disaccharide during emulsification. This diagram shows preparation of a sugar phase using an alternative disaccharide other than honey that does not naturally include hydrogen peroxide.

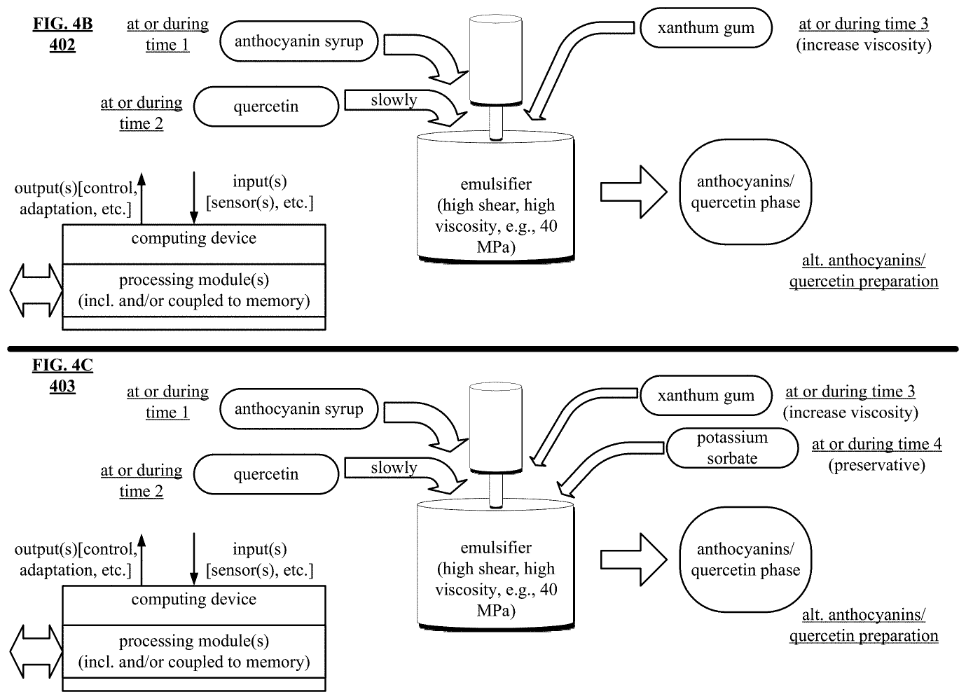

FIGS. 4A, 4B, and 4C are schematic block diagrams of embodiments 401, 402, and 403, respectively, of anthocyanin/quercetin phase preparation in accordance with the present invention.

In certain examples, note that a very high shear, high viscosity emulsifier is employed in each of these Figures FIGS. 4A, 4B, and 4C as described above (e.g., an emulsifier capable of operating at a pressure of 40 mega pascals (MPa) or more is suitable to perform the emulsification operation to generate the anthocyanin/quercetin phase preparation).

Referring to embodiment 401 of FIG. 4A, this diagram shows preparation of an anthocyanin/quercetin phase to be to be used to generate formulations that include anthocyanins and quercetin. In this diagram, anthocyanin syrup is added to the vessel of the emulsifier at or during a first time, and quercetin is added to the vessel of the emulsifier at ordering a second time. Note that such an anthocyanin syrup may be generated from any of a variety of different plant foods that include anthocyanins as described above. In one particular example, the anthocyanin syrup is a BerriQi® syrup, a boysenberry-based anthocyanin syrup. Certain examples of boysenberry-based compositions are described in in U.S. Utility patent application Ser. No. 16/023,327, entitled “BOYSENBERRY COMPOSITIONS AND METHODS OF PREPARATION AND USE THEREOF,” filed Jun. 29, 2018.

In one specific implementation, and anthocyanin syrup having a viscosity of approximately 3,000 mPa-s is used. For example, a BerriQi® syrup having a viscosity of approximately 3,000 mPa-s is used in one particular implementation. Note that the BerriQi® syrup itself includes very small amounts of quercetin because of it being based on a boysenberry extract. Generally speaking, note that any of a very wide variety of plant foods including anthocyanins may be used to provide the anthocyanin syrup. The anthocyanin syrup is generated (e.g., as an extract) from one or more plant foods that includes anthocyanins. Note that certain types of anthocyanin syrups may also inherently include small amounts of quercetin based on the food plant extract used to generate them. In another example, a boysenberry-based anthocyanin syrup other than a BerriQi® syrup having a viscosity of approximately 3,000 mPa-s is used.

To facilitate the combination of the anthocyanin syrup and the quercetin within the emulsifier, the quercetin is slowly added to the anthocyanin syrup that is included within the vessel of the emulsifier. The emulsifier operates on the anthocyanin syrup and the quercetin and produces an even distribution of the anthocyanin syrup and quercetin among one another and also provides for a reduction in particle size of the anthocyanin/quercetin phase. The emulsifier operates on the anthocyanin syrup and quercetin until this even distribution is achieved. In certain examples, emulsification for a period of approximately 10 min. produces an even distribution of the anthocyanin syrup and quercetin among one another and also provides for a reduction in particle size of the anthocyanin/quercetin phase. This is effective as a micronization process that helps to obtain an even distribution and particle size in the formulation as the particle size decreases.

Referring to embodiment 402 of FIG. 4B, this diagram shows an alternative manner of preparation of an anthocyanin/quercetin phase to be used to generate formulations that include anthocyanins and quercetin. This diagram has similarities to the prior diagram but also includes the addition of xanthan gum at or during the third time. The xanthan gum is added as an emulsifying agent to increase the viscosity of the anthocyanin/quercetin phase and to reduce the water availability of the overall emulsion. In certain examples, emulsification for a period of approximately 3-5 min. produces the effect of increased this custody based on need addition of the xanthan gum.

Referring to embodiment 403 of FIG. 4C, this diagram shows an alternative manner of preparation of an anthocyanin/quercetin phase to be used to generate formulations that include anthocyanins and quercetin. This diagram has similarities to the prior diagram but also includes the addition of potassium sorbate at or during a fourth time that is after the third time. Potassium sorbate operates as a preservative and assists to prolong shelf life of formulations that include anthocyanins and quercetin.

Note that and anthocyanin/quercetin phase may be generated based on any of the various embodiments 401, 402, and 403 of FIGS. 4A, 4B, and 4C to be used to generate formulations that include anthocyanins and quercetin. For example, xanthan gum in potassium sorbate need not be added to certain anthocyanin/quercetin phases. For example, for applications in which considerations such an increased viscosity and/or preservative prolongs shelf life are not needed or applicable, an anthocyanin/quercetin phase may be generated based on embodiment 401 of FIG. 4A. However, applications in which considerations such an increased viscosity and/or preservative prolongs shelf life are needed and/or applicable, an anthocyanin/quercetin phase may be generated based on embodiment 402 of FIG. 4B or embodiment 403 of FIG. 4C.

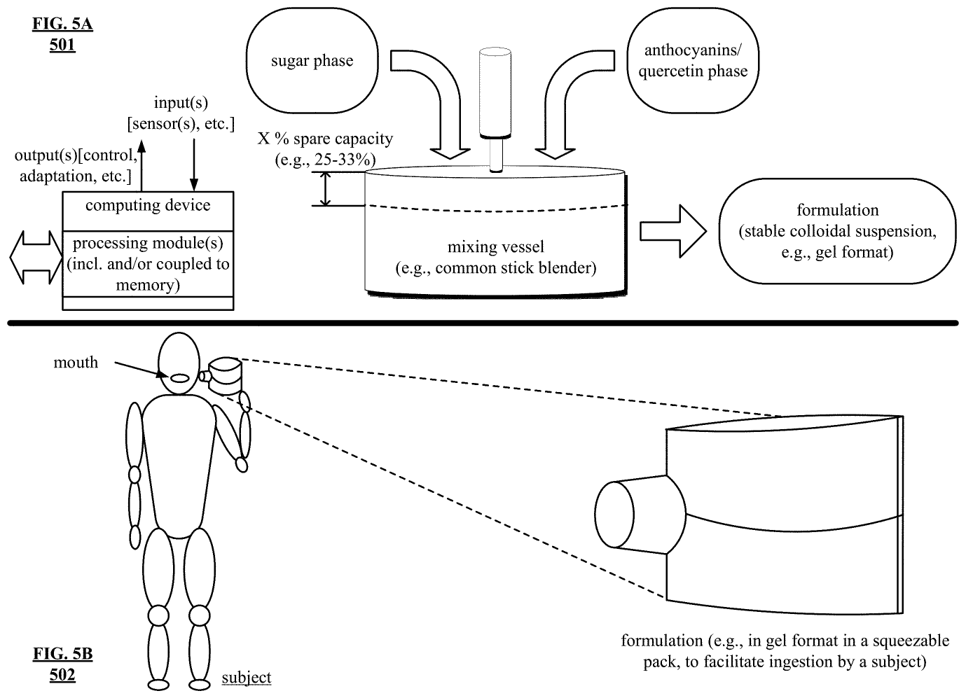

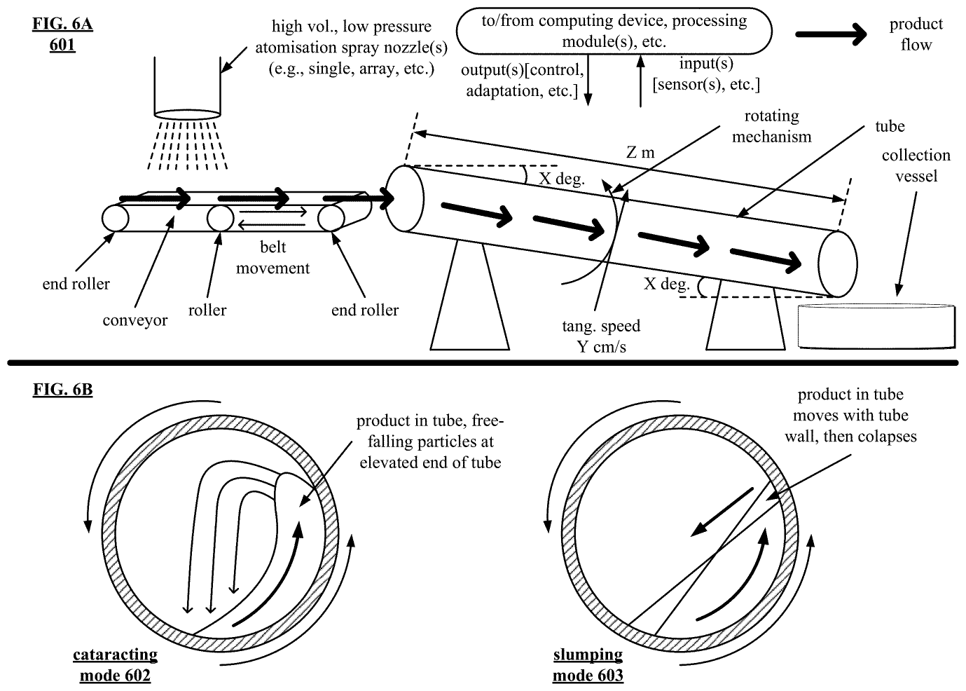

FIG. 5A is a schematic block diagram of an embodiment 501 of a mixing vessel for use to generate formulations in a gel type form based on a sugar phase and an anthocyanin/quercetin phase in accordance with the present invention. A sugar phase and an anthocyanin/quercetin phase are mixed within a mixing vessel. Note that the mixing vessel of this diagram may be configured and implemented to operate in conjunction with a very simple mixer device (e.g., an immersion blender, a common stick blender, a very simplistic and relatively low complexity, low-cost, low-power blender configured to mix the sugar phase and the anthocyanin/quercetin phase, etc.). Any mixing mechanism may be used to combine the sugar phase and the anthocyanin/quercetin phase. In certain examples, a mixing vessel capable holding both the sugar phase in the anthocyanin/quercetin phase together with significant room to spare within the mixing vessel (e.g., 25% to 33% room to spare) is used. The mixing mechanism mixes the sugar phase and the anthocyanin/quercetin phase until a stable colloidal suspension is achieved. This may be viewed as being in a gel format and certain examples.

In an example of operation and implementation, a computing device includes memory (e.g., computer readable memory) that stores operational instructions and one or more processing modules operably coupled to the memory. In certain examples, the computing device is operably coupled to the emulsifier. In certain other examples, the computing device is operably coupled to the mixing vessel.

For example, the one or more processing modules is configured to execute the operational instructions to generate one or more signals (e.g., bit stream, stream, signal sequence, etc. (or their equivalents)) that are provided to the emulsifier to control the operation of the emulsifier (e.g., adjusting the speed of rotation of the emulsifier). In certain examples, the one or more processing modules is configured to execute the operational instructions to generate one or more signals that are provided to the mixing vessel to control the operation of the mixing vessel including the operational speed of the mixing vessel. In addition, note that any of a number of sensors (e.g., speed rotational sensors, pressure sensors, temperature sensors, etc.) may be implemented within the system and operably coupled and configured to provide information to the computing device for use by the computing device in governing the operation of the system (e.g., including the emulsifier and mixing vessel).

In one particular implementation such as using an anthocyanin/quercetin phase on embodiment 401 of FIG. 4A, the amount of the sugar phase comprises approximately 48.32%, the amount of the anthocyanin syrup comprises approximately 48.32%, and the quercetin comprises approximately 3.27% of the total volume of the formulation that includes anthocyanins and quercetin.

In another particular implementation such as using an anthocyanin/quercetin phase based on embodiment 402 of FIG. 4B, the amount of the sugar phase comprises approximately 48.1%, the amount of the anthocyanin syrup comprises approximately 48.1%, the quercetin comprises approximately 3.27%, and the xanthan gum comprises approximately 0.5% of the total volume of the formulation that includes anthocyanins and quercetin.

In yet another particular implementation such as using an anthocyanin/quercetin phase based on embodiment 403 of FIG. 4C, the amount of the sugar phase comprises approximately 47.59%, the amount of the anthocyanin syrup comprises approximately 47.59%, the quercetin comprises approximately 3.22%, the potassium sorbate comprises approximately 0.01%, the olive oil comprises approximately 1%, and the xanthan gum comprises approximately 0.5% of the total volume of the formulation that includes anthocyanins and quercetin. An example of such a formulation in a gel type form including approximately 15.59 g per serving is shown below:

| Ingredients | Amount per 15.59 g serving | % of formulation |
|---|---|---|
| Anthocyanin syrup | 7.42 g | 47.59% |
| Disaccharide | 7.42 g | 47.59% |
| Quercetin | 0.5 g | 3.22% |
| Potassium sorbate | 0.016 g | 0.1% |
| Olive oil | 0.155 g | 1% |
| Xanthan gum | 0.078 g | 0.5% |

Note that the shelf life of such a formulation may be based on a number of different considerations. For example, product handling during manufacture, storage temperature, ambient UV light, humidity, barometric pressure, and/or any of a number of considerations affect the shelf life of such a formulation. Generally speaking, when such a formulation is manufactured in a highly controlled environment using best practice in a Good Manufacturing Practices (GMP) manner (e.g., in accordance with Current Good Manufacturing Practices (CGMP) regulations enforced by the U.S. Food and Drug Administration (FDA)), the microbial load will be minimized significantly, and the formulation shelf life will be extended. In addition, formulations that include potassium sorbate as a preservative will have a longer shelf life.

In even yet another particular implementation such as using an anthocyanin/quercetin phase based on embodiment 403 of FIG. 4C, the amount of the sugar phase comprises approximately 47.84%, the amount of the anthocyanin syrup comprises approximately 47.84%, the quercetin comprises approximately 3.87%, the potassium sorbate comprises approximately 0.01%, the xanthan gum comprises approximately 0.03%, and the ethanol comprises approximately 0.32% of the total volume of the formulation that includes anthocyanins and quercetin. Note that this particular implementation includes no olive oil. An example of such a formulation in a gel type form including approximately 15.59 g per serving is shown below:

| Ingredients | Amount per 15.59 g serving | % of formulation |
|---|---|---|
| Anthocyanin syrup | 7.42 g | 47.84 |
| Disaccharide | 7.42 g | 47.84 |
| Quercetin | 0.6 g | 3.87% |
| Potassium sorbate | 0.015 g | 0.01% |
| Olive oil (none) | 0.0 g | 0% |
| Xanthan gum | 0.005 g | 0.03% |
| Ethanol | 0.05 g | 0.32% |

In yet another particular implementation such as using an anthocyanin/quercetin phase based on embodiment 403 of FIG. 4C, the amount of the sugar phase comprises approximately 12.33%, the amount of the anthocyanin syrup comprises approximately 82.17%, the quercetin comprises approximately 4.93%, the potassium sorbate comprises approximately 0.12%, the xanthan gum comprises approximately 0.04%, and the ethanol comprises approximately 0.41% of the total volume of the formulation that includes anthocyanins and quercetin. An example of such a formulation in a gel type form including approximately 12.16 g per serving is shown below:

| Ingredients | Amount per 12.16 g serving | % of formulation |
|---|---|---|
| Anthocyanin syrup | 10.0 g | 82.17% |
| Disaccharide | 1.5 g | 12.33% |
| Quercetin | 0.6 g | 4.93% |
| Potassium sorbate | 0.015 g | 0.12% |
| Xanthan gum | 0.005 g | 0.04% |
| Ethanol | 0.05 g | 0.41% |

In certain examples of preparing a formulation as described herein, hurdle technology is employed to facilitate better food preservation and extended shelf life. For example, based on manufacturing the formulation in a highly controlled environment as described above (e.g., CGMP regulations enforced by the U.S. FDA), the microbial load is significantly minimized, and the formulation shelf life is extended.

In certain instances, the following hurdle technology methodology is used:

(acidity−pH)+(water activity(a w))+(preservative)+(redox potential (simplistically oxidative stress))+/−(temperature(t),refrigeration)=x shelf life

Water activity (a w) 0.85 or less

Potassium sorbate of 0.10% by volume of finished product (1000 mg/liter)

pH of 3.8 or below

Syringe or GU packaging stored without the presence of oxygen

Storage in a UV light stable environment

Refrigerate in storage, and put a ‘please refrigerate’ on the label of the package before delivery to a customer.

With this hurdle technology methodology used to improve preservation and extend shelf life, a shelf life stability of 6 or more months may be realized and a shelf life stability of 24 or more months may be realized when the formulation is accompanied with refrigeration. In addition, note that manufacturing such a formulation in a clean and stable environment also operates to maximize shelf life.

In certain other instances, the following hurdle technology methodology is used:

(acidity−pH)+(water activity(a w))+(preservative)+(redox potential (simplistically oxidative stress))+/−(temperature(t),refrigeration)=x shelf life

Water activity (a w) 0.65 or less

Potassium sorbate of 0.015% by volume of finished product (1000 mg/liter)

pH of 3.8 or below

10 dose 100 ml bottle or single shot GU packaging stored without the presence of oxygen

Storage in a UV light stable environment

Refrigerate in storage, and put a ‘please refrigerate’ on the label of the package before delivery to a customer.

With this alternative hurdle technology methodology used to improve preservation and extend shelf life, the shelf life stability of even more than 6 months may be realized and a shelf life stability of even more than 24 months may be realized when the formulation is accompanied with refrigeration. In addition, as mentioned above, note that manufacturing such a formulation in a clean and stable environment also operates to maximize shelf life.

FIG. 5B is a schematic block diagram of an embodiment 502 of a subject ingesting a formulation in accordance with the present invention. This diagram shows a formulation that is manufactured as described herein and is packaged for distribution and to facilitate consumption by a subject (e.g., alternatively referred to as a user, a person, a patient, a human, etc. or equivalent nomenclature). For example, the formulation is included within a packaging similar to a squeezable energy pack that includes the formulation in a gel type form. The subject removes the cap of the packaging and squeezes the formulation into the subject's mouth and ingests the formulation. In certain limitations, each packaging of formulation would include one serving of the formulation, such as an approximate 15.59 g amount per serving as described above with respect to certain embodiments.

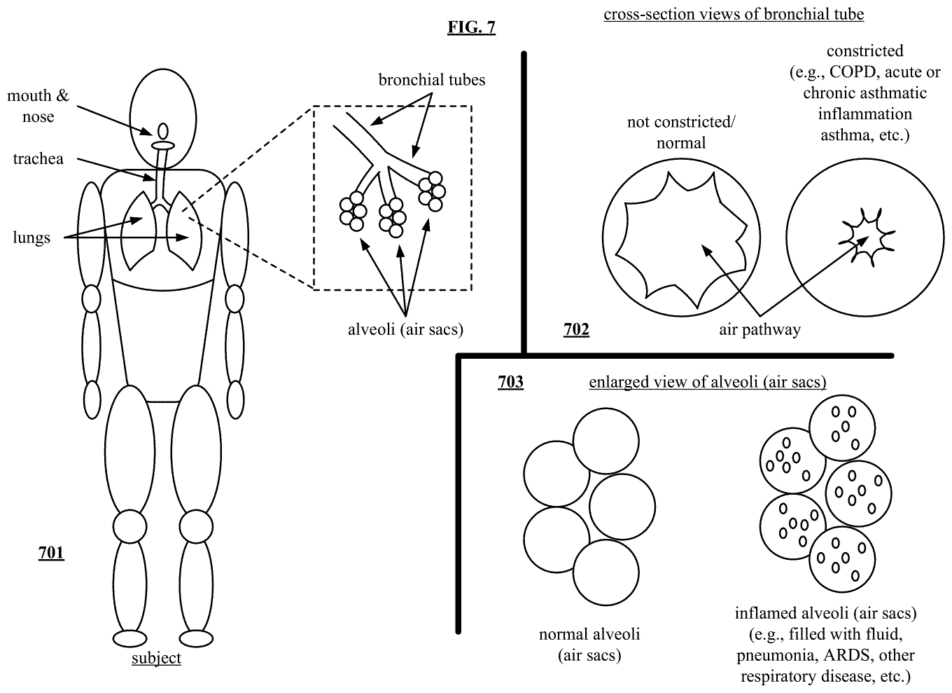

FIG. 6A is a schematic block diagram of an embodiment 601 of a system for use to generate formulations in a dry type form based on a sugar phase and an anthocyanin/quercetin phase in accordance with the present invention. A formulation having a dry format may be administered to a subject in a variety of forms. The formulation in a dry type form may be included within the capsule that is swallowed by a subject for absorption via the liver into the bloodstream of the subject. Alternatively, the formulation in a dry type form may be administered via inhalation into the lungs of the subject for absorption into the lining of the lungs and into the bloodstream of the subject.

Referring to embodiment 601, a disaccharide and water-based solution is sprayed using one or more high-volume, low pressure atomization spray nozzles. Certain limitations include a single high-volume, low pressure atomization spray nozzle, and others include multiple high-volume, low pressure atomization spray nozzles such as in an array implementation, such as including multiple spray nozzles arranged in a matrix (e.g., a matrix of X×Y spray nozzles arranged along in a square or rectangular pattern, where X and Y are each positive integers greater than or equal to 2), or in a line (e.g., X number spray nozzles arranged along an axis, where X is a positive integer greater than or equal to 2). In a specific example, a spray of a 60% sucrose to water solution is sprayed onto an anthocyanin/quercetin mix that is spread out on a conveyor belt system. For example, the conveyor belt system may include a belt that is approximately 3 mm thick and is configured to transport the combined sucrose to water solution and anthocyanin/quercetin mix to a hollow tube or cylinder that includes a mechanism to rotate it with the sucrose to water solution and anthocyanin/quercetin mix in it.

The hollow tube or cylinder is angled downward such that the sucrose to water solution and anthocyanin/quercetin mix, as it travels downward within the hollow tube or cylinder as the hollow tube as the hollow tube or cylinder is rotating, the sucrose to water solution and anthocyanin/quercetin mixed together before being deposited within a collection vessel. Note that the particular downward angular alignment and the length of the hollow tube or cylinder may be different as desired in different implementations. In one specific implementation, the downwardly angular alignment of the hollow tube or cylinder is X degrees below horizontal (e.g., where X is an angular degree within the range of 2° and 10°; one specific implementation includes an angular alignment of X being 4°), and the length of the hollow tube or cylinder is Z (e.g., such as measured in meters where is Z is a number between 2 and 7 such that the length of the hollow tube or cylinder is some desired length between 2 m and 7 m; one specific implementation includes a hollow tube or cylinder that is 4 m in length).

Note that the diameter of the hollow tube or cylinder and the width of the conveyor belt may be appropriately selected so they are approximately similar in size. For example, consider a hollow tube or cylinder that is approximately 400 mm in diameter, then the width of the conveyor belt is selected so as to facilitate delivery of the sucrose to water solution and anthocyanin/quercetin mix to the hollow tube or cylinder so that it may be next as it is traveling down through the hollow tube or cylinder towards the collection vessel. Note that the rotational speed of the hollow tube or cylinder is fast enough such as to facilitate a cataract mode of operation so that the sucrose to water solution and anthocyanin/quercetin mix is effectively next. The rotational speed of the hollow tube or cylinder should not be slow enough so as to facilitate a slumping mode of operation when mixing the sucrose to water solution and anthocyanin/quercetin mix. Such modes of a product within a hollow tube or cylinder that is rotating are described below.

FIG. 6B is a schematic block diagram showing different examples 602 and 603, respectively, of modes of a product within a rotating tube or cylinder for use to generate formulations in a dry type form in accordance with the present invention.

Referring to embodiment 602 showing a cataracting mode of a product within a tube or cylinder, a cataracting mode is achieved when the hollow tube or cylinder is operating at half sufficiently high rotational speed such that the product includes freefalling particles at the elevated end of the cylinder. For example, consider that the tube or cylinder is rotating counterclockwise as shown in this diagram, then the product in the hollow tube or cylinder free falls back to the bottom of the hollow tube or cylinder after it is carried up the right-hand side wall of the tube or cylinder that is rotating counterclockwise. This cataracting mode is a preferred manner to perform mixing of the sucrose to water solution and anthocyanin/quercetin mix.

Referring to embodiment 603 showing a slumping mode of a product within a rotating tube or cylinder, when the rotational speed of the hollow tube or cylinder is insufficient to facilitate a cataracting mode, the product tends to move up the right-hand side wall of the tube or cylinder that is rotating counterclockwise and then, at some particular elevation, the product suddenly collapses and moves back towards its initial position. In certainties and put, this slumping mode of operation is inadequate to perform mixing of the sucrose to water solution and anthocyanin/quercetin mix.

Referring back to the embodiment 602 of FIG. 6A, one manner of preparing a formulation in a dry type form is described. A sucrose to water solution is sprayed via the one or more high-volume, low pressure atomization spray nozzles onto the conveyor belt system. For example, a 60% sucrose to water solution is sprayed onto an anthocyanin/quercetin mix that is spread out on the conveyor belt system. The conveyor belt system transports the sucrose to water solution and anthocyanin/quercetin mix to a hollow tube or cylinder (e.g., 4 m in length, 400 mm in diameter) that is driven using a rotating mechanism at a sufficient rate so as to allow a cataracting mode of operation and not allow a slumping motion mode of operation.

In an example of operation and implementation, a computing device includes memory (e.g., computer readable memory) that stores operational instructions and one or more processing modules operably coupled to the memory. In certain examples, the computing device is operably coupled to the rotating mechanism (e.g., an electric motor) and configured to control the operation of the rotating mechanism including the rotational speed of the hollow tube or cylinder. In certain other examples, the computing device is operably coupled to the conveyor belt and the atomization spray nozzles and configured to control the operation of them as well.

For example, the one or more processing modules is configured to execute the operational instructions to generate one or more signals (e.g., bit stream, stream, signal sequence, etc. (or their equivalents)) that are provided to the rotating mechanism to control the operation of the rotating mechanism (e.g., adjusting the speed of rotation of the hollow tube or cylinder to facilitate a cataracting mode of operation and not allow a slumping motion mode of operation). In certain examples, the one or more processing modules is configured to execute the operational instructions to generate one or more signals that are provided to the conveyor belt to control the operation of the conveyor belt including the operational speed of the conveyor belt. Also, in certain other examples, the one or more processing modules is configured to execute the operational instructions to generate one or more signals that are provided to the atomization spray nozzles to control the operation of the atomization spray nozzles including the volume of material sprayed through the atomization spray nozzles, the pressure by which the material is sprayed through the atomization spray nozzles, etc. In addition, note that any of a number of sensors (e.g., speed rotational sensors, pressure sensors, temperature sensors, etc.) may be implemented within the system and operably coupled and configured to provide information to the computing device for use by the computing device in governing the operation of the system.

In one specific implementation, the downward angular alignment of the hollow tube or cylinders is approximately a 4° down angle, and a tangential speed of the hollow tube or cylinder when rotating is within the 10-15 cm/s range (e.g., to facilitate pilling of the anthocyanin/quercetin mix as it travels down the hollow tube or cylinder). In addition, note that the rotational speed of the hollow tube or cylinder may be adjusted in real time based on how well the sucrose to water solution and anthocyanin/quercetin mix is mixing. For example, if the rotational speed is too slow and a slumping mode of operation begins to occur, the rotational speed is increased until a cataracting mode of operation resumes. In certain examples, based on mixing in accordance with a cataracting mode of operation, the finished product/formulation being deposited from the end of the hollow tube or cylinder into the collection vessel includes small orb shaped conglomerates approximately 3-4 mm in size.

After the formulation has dried, it may be packaged in any of a number of desired ways. For example, the formulation is packaged in capsules that are distributed for consumption by one or more subjects. In another example, the formulation is packaged for a bronchial type inhaler such that the dry type form is administered via inhalation by a subject and absorbed into the subject's bloodstream via the internal lining of the lungs of the subject.

An example of such a formulation in a dry type form including approximately 4.1 g per serving is shown below:

| Ingredients | Amount per 4.1 g serving | % of formulation |
|---|---|---|
| Anthocyanin powder | 2.1 g | 51.2% |
| Quercetin powder | 0.5 g | 12.2% |
| 60% sucrose to | 1.5 g | 36.6% |
| water solution | | |

FIG. 7 is a schematic block diagram showing examples 701, 702, and 703, respectively, of portions of a respiratory system of a subject that may be adversely affected by one or more conditions in accordance with the present invention.

Referring to example 701, certain portions of the respiratory system of a subject are shown. For example, when breathing, the user takes in air via the nose office action mouth, and the air travels down the trachea and into the lungs. The lungs include bronchial tubes (alternatively referred to as bronchi, including both large and medium-sized air pathways) that are terminated with alveoli. These alveoli are air sacs within the lungs that facilitate gas exchange (e.g., carbon dioxide and oxygen) and also the oxygenation of blood during operation of the cardiovascular and respiratory systems of the subject as the subject breathes. The alveolar membrane of the alveoli is the gas exchange surface via which the gas exchange (e.g., carbon dioxide and oxygen) is performed. Also, the alveolar membrane is surrounded by a network of capillaries that facilitate the oxygenation of the blood of the subject and the removal of carbon dioxide. As the subject breathes, air containing oxygen travels into the lungs and to the alveoli, the oxygen is diffused into these capillaries, and carbon dioxide is released from the capillaries into the alveoli and is breathed out by the subject. The alveoli are the smallest functional units in the respiratory tract of the subject. The diameter of a single air sac or alveolus is typically within the range of 200 to 500 μm, and the subject's lungs typically will contain approximately 300 million alveoli. This very large number of alveoli corresponds to a very large surface area by which the gas exchange (e.g., carbon dioxide and oxygen) is performed. For example, this may correspond to a surface area within the range of 700 to 800 sq. ft. within a subject. Various ailments may adversely affect the health of a subject and specifically adversely affect the operation of the respiratory system of the subject. Examples of such ailments may include any one or more of asthma, pneumonia, interstitial lung disease, acute respiratory distress syndrome (ARDS), chronic obstructive pulmonary disorder (COPD), bronchitis, autoimmune lung conditions, etc.

Referring to example 702, cross-sectional views of bronchial tube is shown. On the left-hand side, the bronchial tube is shown as not being constricted or in a normal state such that the air pathway between the trachea and the alveoli is not constricted and thereby facilitates the free movement of air into the lungs and to the alveoli. On the right-hand side, the bronchial tube is shown as being constricted, such as in response to any of a number of different ailments including those described above, such that the air pathway is significantly reduced, which can reduce result in respiratory distress for the subject. For example, when the bronchial tubes become inflamed and/or swollen, the subject may suffer from breathing problems including any one or more of chest congestion such that the subject's chest feels tight, full, clog, etc., mucus buildup within the lungs that may be of a variety of different colors such as clear, white, yellow, or green, shortness of breath, wheezing due to the constricted bronchial tubes of the subject, etc.

Referring to example 703, an enlarged view of alveoli (air sacs) on the subject are shown. On the left-hand side, the alveoli are shown as being in a normal state such that they can function properly to facilitate gas exchange (e.g., carbon dioxide and oxygen) and also the oxygenation of blood during operation of the cardiovascular and respiratory systems of the subject as the subject breathes. On the right-hand side, the alveoli are shown as being inflamed and not being fully functional to facilitate gas exchange (e.g., carbon dioxide and oxygen) and also the oxygenation of blood during operation of the cardiovascular and respiratory systems of the subject as the subject breathes. Certain ailments, such as pneumonia, Tillable alveoli of the subject's lungs with fluid thereby limiting the amount of oxygen that is diffused into the subject blood and the amount of carbon dioxide that is removed from the auctions blood during breathing.

A formulation based on a combination of quercetin and anthocyanins as described herein, when administered to a subject, improves the respiratory health of the subject. For example, such a formulation reduces the inflammation with in the lungs. The health benefits of reducing inflammation within the lungs, such as reducing constriction of the bronchial tubes and reducing inflammation of the alveoli, provides improvement in the operation of the respiratory system of the subject. In addition, such a formulation, when administered to a subject, is operative to facilitate and ease the removal of mucus from the lungs of the subject. Moreover, such a formulation, when administered to a subject who may be suffering from a virus-based ailments, is operative to stop or reduce viral replication of the virus itself within the lungs of the subject. For example, consider the subject who has been exposed to an infected by a virus (e.g., a virus associated with the common cold, the severe acute respiratory syndrome coronavirus 2 (SARS-CoV-2), and/or some other virus). Stopping or reducing viral replication within the subject can also reduce the severity and duration of such an ailments and provide improved health to the subject. Stopping or reducing viral replication within the subject is one of the great challenges in stemming the deleterious effects of such an infection.

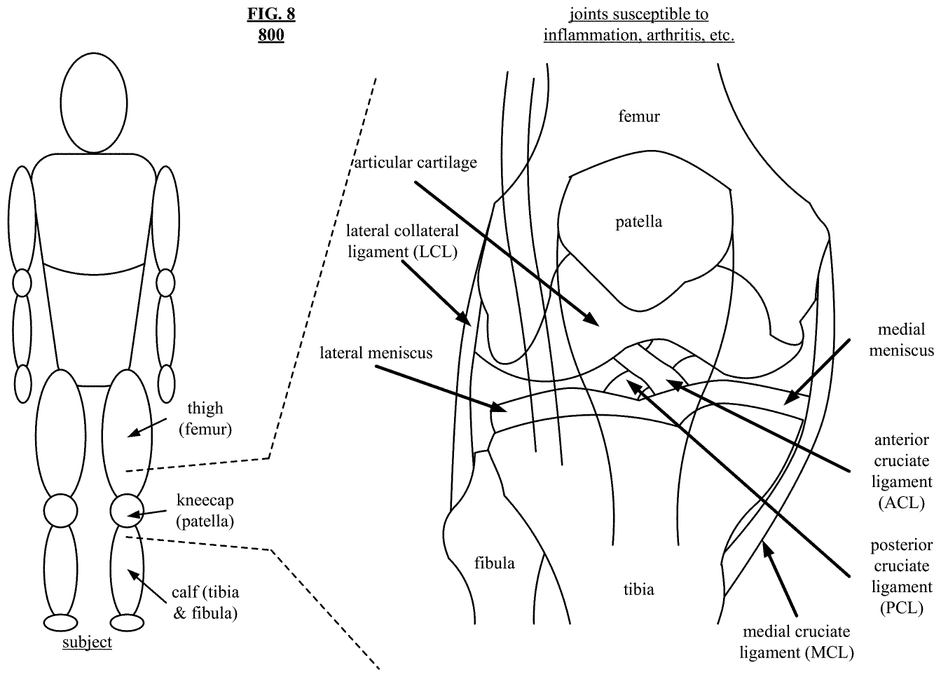

FIG. 8 is a schematic block diagram showing an example 800 of portions of a knee joint of a subject that may be adversely affected by one or more conditions in accordance with the present invention. This diagram shows several of the major components of a knee joint of the subject. At the knee joint of a subject, the femur (i.e., the thighbone between the knee and the hip) is connected to the two bones, namely, tibia and fibula, of the lower portion of the leg. In addition, the knee joint includes the patella, the knee at the front of the knee of the subject. In addition, there are certain soft tissues including ligaments that connect the bones at the knee joint of the subject.

There are four major ligaments in the knee, which serve as connecting tissue that connects bones to another and provide stability and strength to the knee joint of the subject. The anterior cruciate ligament (ACL) is located in the center of the knee, and it controls rotation and forward movement of the tibia. The posterior cruciate ligament (PCL) is located in the back of the knee, and it controls backward movement of the tibia. The medial collateral ligament (MCL) is located on the side of the knee, opposite the fibula, and it gives stability to the inter knee. The lateral collateral ligament (LCL) is located on the other side of the knee, on the same side as the fibula, and it gives stability to the outer knee.

In addition, there are other soft tissues included within the knee joint of the subject. For example, at the top of the tibia is lateral meniscus and medial meniscus that provide for and facilitate the interfacing between the femur and tibia. Also, articular cartilage, at the bottom of the femur, also helps to provide for and facilitate the interfacing between the femur and tibia.

Consider an injury occurring to one or more components of the knee joint of the subject. When such an injury happens, one or more of the bones associated with the knee joint may be broken and/or one or more of the ligaments, soft tissues, etc. associated with the knee joint of the subject made be torn, damaged, injured, etc. In many instances, injury to one or more of the ligaments, soft tissues, etc. results in inflammation of those one or more of the ligaments, soft tissues, etc. In addition, other ailments may adversely affect one or more components of the knee joint of the subject. For example, arthritis is inflammation within one or more joints of the subject. Consider this diagram, arthritis within the knee joint of the subject would be inflammation of one or more of the ligaments, soft tissues, etc. of the knee joint of the subject. Symptoms of arthritis may include pain, stiffness, limited function such as loss of range of movement of the joint, redness of the skin around the joints, swelling, weakness of the joint, etc. Two common forms of arthritis are osteoarthritis (e.g., such as associated with wear and tear of cartilage within a joint of the subject) and rheumatoid arthritis (e.g., such as associated with inflammation of one or more components of a joint of the subject based on inflammation such as being caused by a misdirected immune system). Note also that there are other types of arthritis including psoriatic arthritis (e.g., such as chronic disease by characterized by inflammation of the skin/psoriasis and joints), reactive arthritis (e.g., such as inflammation of one or more joints of the subject due to an infection in another part of the subject), infectious arthritis (e.g., such as arthritis caused by an infection of one or more joints by microorganisms), metabolic arthritis (e.g., such as gout, which is often caused by that diet of a subject), among others.

A formulation based on a combination of quercetin and anthocyanins as described herein, when administered to a subject, reduces the inflammation within one or more portions of the subject. As described above, reduction in inflammation within the lungs including the bronchial tubes of the lungs of the subject improves the respiratory health of the subject. In addition, when a subject is suffering from inflammation within one or more joints, such as the knee joint as shown in this diagram, when such a formulation as described herein is administered to the subject, the inflammation within the one or more joints of the subject is reduced thereby improving the joint health of the subject. Also, when the subject is suffering from arthritis, when such a formulation as described herein is administered to the subject, the symptoms of the arthritis are reduced thereby improving the joint health of the subject.

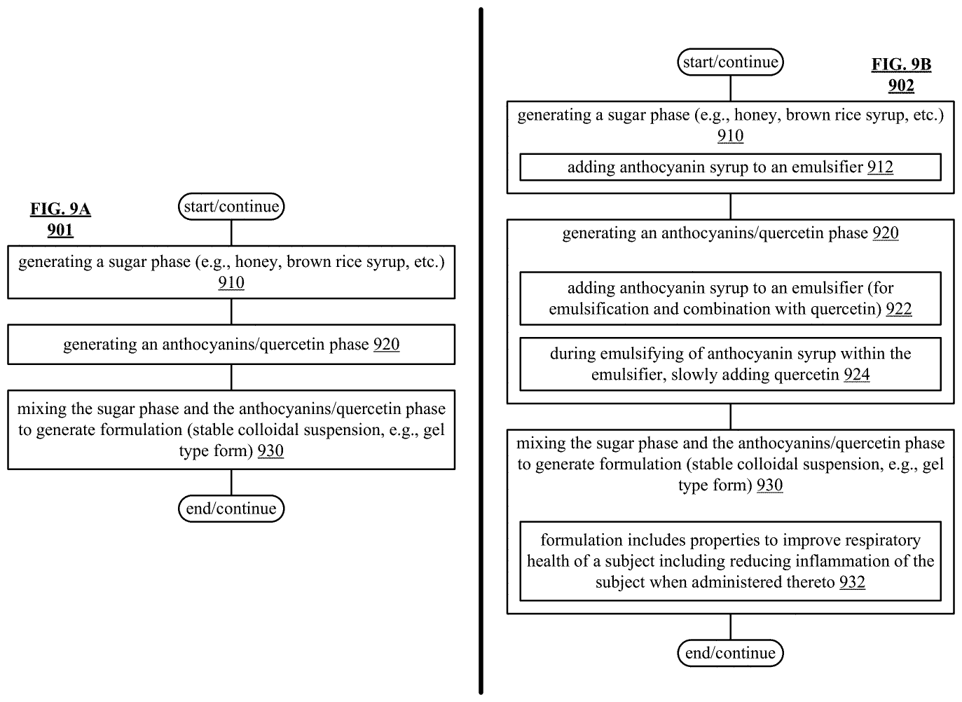

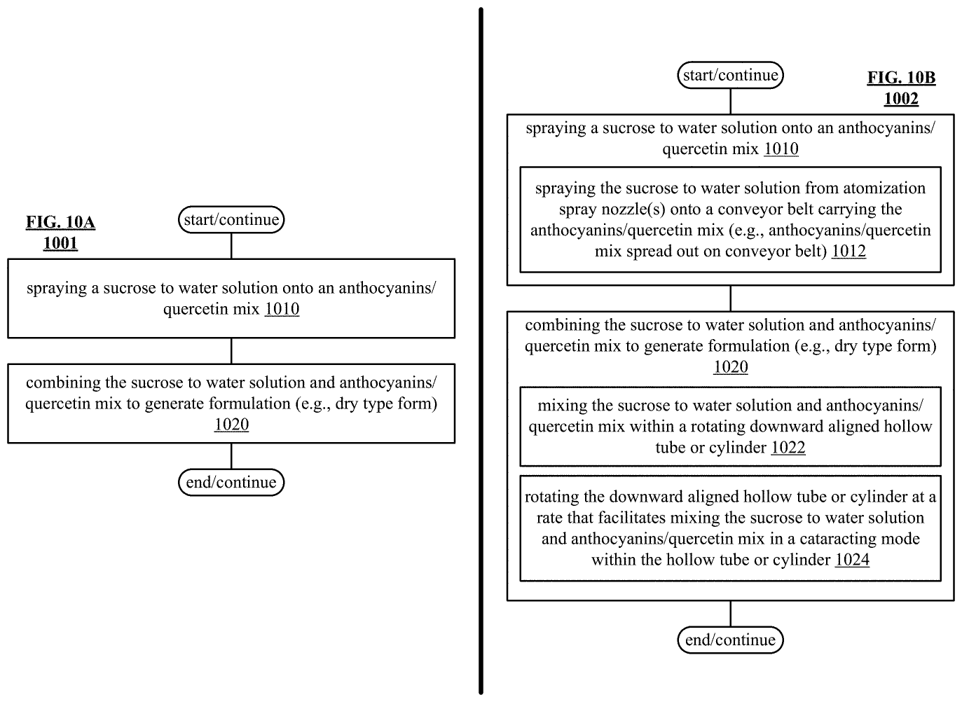

FIGS. 9A, 9B, 10A, and 10B are schematic block diagrams of embodiments of methods for execution to by one or more devices to generate a formulation in accordance with the present invention.

Referring to the method 901 of FIG. 9A, the method 901 operates in step 910 by generating a sugar phase (e.g., honey, brown rice syrup, etc.). The method 900 continues in step 920 by generating an anthocyanin/quercetin phase. The method 900 also operates in step 930 by mixing the sugar phase and the anthocyanin/quercetin phase to generate formulation (stable colloidal suspension, e.g., gel type form).

Referring to the method 902 of FIG. 9B, the method 902 is a variant of the method 901 of FIG. 9A.

Referring to the method 902 of FIG. 9A, the method 902 operates in step 910 by generating a sugar phase (e.g., honey, brown rice syrup, etc.). In addition, the method 902 also operates in step 912 by adding anthocyanin syrup to an emulsifier. The method 902 continues in step 920 by generating an anthocyanin/quercetin phase. In addition, the method 902 also operates in step 920 by adding anthocyanin syrup to an emulsifier (for emulsification and combination with quercetin). Also, the method 902 operates in step 924 by slowly adding quercetin during emulsifying of anthocyanin syrup within the emulsifier.

The method 902 also operates in step 930 by mixing the sugar phase and the anthocyanin/quercetin phase to generate formulation (stable colloidal suspension, e.g., gel type form). As shown in the block/step 932, the formulation includes properties to improve respiratory health of a subject including reducing inflammation of the subject when administered to the subject.

Referring to the method 1001 of FIG. 10A, the method 1001 operates in step 1010 by generating a sugar phase (e.g., honey, brown rice syrup, etc.). The method 1001 operates in step 1010 by spraying a sucrose to water solution onto an anthocyanin/quercetin mix. The method 1001 continues in step 1020 by combining the sucrose to water solution and anthocyanin/quercetin mix to generate formulation (e.g., dry type form).

Referring to the method 1002 of FIG. 10B, the method 1002 is a variant of the method 1001 of FIG. 10A. The method 1002 operates in step 1010 by spraying a sucrose to water solution onto an anthocyanin/quercetin mix. In addition, the method 1002 operates in step 1012 by spraying the sucrose to water solution from atomization spray nozzle(s) onto a conveyor belt carrying the anthocyanin/quercetin mix (e.g., anthocyanin/quercetin mix spread out on conveyor belt).

The method 1002 continues in step 1020 by combining the sucrose to water solution and anthocyanin/quercetin mix to generate formulation (e.g., dry type form). In addition, the method 1002 operates in step 1022 by

Also, the method 1002 operates in step 1024 by mixing the sucrose to water solution and anthocyanin/quercetin mix within a rotating downward aligned hollow tube or cylinder. The method 1002 operates in step 1040 by rotating the downward aligned hollow tube or cylinder at a rate that facilitates mixing the sucrose to water solution and anthocyanin/quercetin mix in a cataracting mode within the hollow tube or cylinder.

As may be used herein, the terms “substantially” and “approximately” provides an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provides an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, a quantum register or other quantum memory and/or any other device that stores data in a non-transitory manner. Furthermore, the memory device may be in a form of a solid-state memory, a hard drive memory or other disk storage, cloud memory, thumb drive, server memory, computing device memory, and/or other non-transitory medium for storing data. The storage of data includes temporary storage (i.e., data is lost when power is removed from the memory element) and/or persistent storage (i.e., data is retained when power is removed from the memory element). As used herein, a transitory medium shall mean one or more of: (a) a wired or wireless medium for the transportation of data as a signal from one computing device to another computing device for temporary storage or persistent storage; (b) a wired or wireless medium for the transportation of data as a signal within a computing device from one element of the computing device to another element of the computing device for temporary storage or persistent storage; (c) a wired or wireless medium for the transportation of data as a signal from one computing device to another computing device for processing the data by the other computing device; and (d) a wired or wireless medium for the transportation of data as a signal within a computing device from one element of the computing device to another element of the computing device for processing the data by the other element of the computing device. As may be used herein, a non-transitory computer readable memory is substantially equivalent to a computer readable memory. A non-transitory computer readable memory can also be referred to as a non-transitory computer readable storage medium.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A formulation comprising:
anthocyanins; and
quercetin.

2. The formulation of claim 1 further comprising:
anthocyanin syrup, wherein the anthocyanin syrup includes the anthocyanins.

3. The formulation of claim 1 further comprising:
a sugar phase; and
an anthocyanin/quercetin phase that includes the anthocyanins and the quercetin.

4. The formulation of claim 3, wherein the sugar phase is based on honey or brown rice syrup.

5. The formulation of claim 3, wherein the sugar phase is configured to facilitate absorption of the quercetin into a subject based on the formulation being administered to the subject.

6. The formulation of claim 3, wherein:
the sugar phase is generated based on an emulsification process applied to a disacharide; and
the sugar phase is configured to facilitate combination of the anthocyanins and the quercetin including to facilitate distribution of the anthocyanins and the quercetin within the formulation.

7. The formulation of claim 3, wherein:
the sugar phase is generated based on an emulsification process applied to a disacharide and olive oil;
the sugar phase is configured to facilitate combination of the anthocyanins and the quercetin including to facilitate distribution of the anthocyanins and the quercetin within the formulation; and
the olive oil is configured to eliminate or reduce effect on the anthocyanins and the quercetin based on hydrogen peroxide within the disacharide.

8. The formulation of claim 3, wherein:
the anthocyanin/quercetin phase is generated based on an emulsification process applied to an anthocyanin syrup and the quercetin, wherein the anthocyanin syrup includes the anthocyanins.

9. The formulation of claim 3, wherein:
the anthocyanin/quercetin phase is generated based on an emulsification process applied to an anthocyanin syrup, the quercetin, and at least one of xantham gum and potassium sorbate, wherein the anthocyanin syrup includes the anthocyanins.

10. The formulation of claim 1, wherein the formulation includes a gel type form.

11. The formulation of claim 1, wherein the formulation includes a dry type form.

12. The formulation of claim 1, wherein, based on the formulation being administered to a subject, the formulation is configured to perform at least one of increase elasticity of lungs of the subject, reduce of inflammation within lungs of the subject, minimize buildup of fluid within the lungs of the subject, and facilitate removal of mucus from the lungs of the subject, and reduce pneumonia of the subject thereby improving respiratory function of the lungs of the subject.

13. The formulation of claim 1, wherein, based on the formulation being administered to a subject, the formulation is configured to stop or reduce viral replication within the subject.

14. The formulation of claim 1, wherein the anthocyanins are extracted from a food plant, wherein the food plant includes at least one of boysenberries, blueberries, raspberries, cranberries, black raspberries, red raspberries, and blackcurrants, Marion blackberries, wild blueberries, cherries, redcurrant, purple corn, blue corn, red grapes, red cabbage, red-flashed peaches and apples contain anthocyanins.

15. A formulation comprising:
a sugar phase; and
an anthocyanin/quercetin phase that includes anthocyanins and quercetin.

16. The formulation of claim 15, wherein:
the sugar phase is generated based on an emulsification process applied to a disacharide;
the sugar phase is configured to facilitate combination of the anthocyanins and the quercetin including to facilitate distribution of the anthocyanins and the quercetin within the formulation; and
the anthocyanin/quercetin phase is generated based on another emulsification process applied to an anthocyanin syrup and the quercetin, wherein the anthocyanin syrup includes the anthocyanins.

17. The formulation of claim 15, wherein:
the sugar phase is generated based on the emulsification process applied to a disacharide and also to olive oil;
the sugar phase is configured to facilitate combination of the anthocyanins and the quercetin including to facilitate distribution of the anthocyanins and the quercetin within the formulation;
the olive oil is configured to eliminate or reduce effect on the anthocyanins and the quercetin based on hydrogen peroxide within the disacharide; and
the anthocyanin/quercetin phase is generated based on the another emulsification process applied to an anthocyanin syrup, the quercetin, and also to at least one of xantham gum and potassium sorbate, wherein the anthocyanin syrup includes the anthocyanins.

18. The formulation of claim 15, wherein, based on the formulation being administered to a subject, the formulation is configured to perform at least one of increase elasticity of lungs of the subject, reduce of inflammation within lungs of the subject, minimize buildup of fluid within the lungs of the subject, and facilitate removal of mucus from the lungs of the subject, reduce pneumonia of the subject thereby improving respiratory function of the lungs of the subject, and stop or reduce viral replication within the subject.

19. A formulation comprising:
a sugar phase; and
an anthocyanin/quercetin phase that includes anthocyanins and quercetin, wherein:
the sugar phase is generated based on an emulsification process applied to a disacharide;
the sugar phase is configured to facilitate combination of the anthocyanins and the quercetin including to facilitate distribution of the anthocyanins and the quercetin within the formulation; and
the anthocyanin/quercetin phase is generated based on another emulsification process applied to an anthocyanin syrup and the quercetin, wherein the anthocyanin syrup includes the anthocyanins;
based on the formulation being administered to a subject, the formulation is configured to perform at least one of increase elasticity of lungs of the subject, reduce of inflammation within lungs of the subject, minimize buildup of fluid within the lungs of the subject, and facilitate removal of mucus from the lungs of the subject, reduce pneumonia of the subject thereby improving respiratory function of the lungs of the subject, and stop or reduce viral replication within the subject; and
the formulation includes a gel type form or a dry type form.

20. The formulation of claim 19, wherein:
the sugar phase is generated based on the emulsification process applied to the disacharide and also to olive oil;
the olive oil is configured to eliminate or reduce effect on the anthocyanins and the quercetin based on hydrogen peroxide within the disacharide; and
the anthocyanin/quercetin phase is generated based on the another emulsification process applied to the anthocyanin syrup, the quercetin, and also to at least one of xantham gum and potassium sorbate.
